(19) United States
(12) Patent Application Publication
(10) Pub. No.: US 2006/0189897 A1
(43) Pub. Date: Aug. 24, 2006

(54) Polymer jacket for a guidewire

(76) Inventors: Philippe Poncet, Sandy Hook, CT (US); James Visteur Dandeneau, Thompson, CT (US)

Correspondence Address: CANTOR COLBURN, LLP, 55 GRIFFIN ROAD SOUTH, BLOOMFIELD, CT, 06002, US

(21) Appl. No.: 11/338,192

(22) Filed: Jan. 23, 2006

Related U.S. Application Data
(60) Provisional application No. 60/645,626, filed on Jan. 21, 2005.

(51) Int. Cl. A61M 25/00

(52) U.S. Cl. 600/585

(57) ABSTRACT

Disclosed herein is a guidewire comprising a core; and a jacket disposed upon the core; wherein the jacket comprises a segmented layer that is radially disposed upon the core and wherein the segmented layer comprises at least two segments having different compositions and/or different physical properties. Disclosed herein too is a method of manufacturing a guidewire comprising extruding onto a core a jacket, wherein the jacket comprises a segmented layer that is radially disposed upon the core and wherein the segmented layer comprises at least two segments having different compositions and/or different physical properties.

### CROSS REFERENCE TO RELATED APPLICATIONS

This application claims priority to U.S. Provisional Application Ser. No. 60/645,626 filed on Jan. 21, 2005, which is incorporated in its entirety by reference herein.

### BACKGROUND

This disclosure relates to a polymer jacket disposed upon a guidewire. In particular, this disclosure relates to a polymer jacket having a variable stiffness along its longitudinal axis disposed upon a guidewire.

Guidewires are generally inserted in the body through sheaths, needles or introducers and are generally used in the treatment of a diseased segment of a blood vessel (e.g., artery) or other body lumen (e.g., bile duct) by providing a guiding track for devices such as, for example, a catheter, a stent delivery system, a retrieval basket, or the like, to the point of application inside the blood vessel or the said lumen. Guidewires used in neurovascular, cardiovascular, endovascular and endoscopic procedures are about 0.22 to 1.00 millimeters (mm) in diameter and usually exceed 1,000 millimeters in length.

A guidewire can commonly comprise a core and a jacket. It is also desirable for the guidewire to permit flexibility during manufacturing to allow for different shapes, sizes, stiffnesses, or the like. Materials commonly used for the guidewire core itself include alloys such as, for example, stainless steels, cobalt-chromium (CoCr) alloys, nitinol, alloys of nitinol with other ternary elements, polymers, polymeric composites, or the like. The guidewire core can be manufactured from or incorporate a radio opaque material to facilitate imaging (using xrays) during the treatment of a desired diseased blood vessel or lumen. The core is generally circular in cross section and can be manufactured from wire, tubing, or combinations thereof. Additionally, the cross sectional area of the guidewire core is usually smaller at the distal end of the guidewire than at the proximal end to allow for adequate floppiness and atraumatic features.

It is generally desirable for the guidewire to be flexible, but yet to be able to transmit a linear and/or a rotary force from the proximal end to the distal end in order to navigate to the diseased segment thru anatomic tortuosity. Guidewires that are currently commercially available do not permit the range of flexibility that can be advantageously used by a surgeon during application.

### SUMMARY

Disclosed herein is a guidewire comprising a core; and a jacket disposed upon the core; wherein the jacket comprises a segmented layer that is radially disposed upon the core and wherein the segmented layer comprises at least two segments having different compositions and/or different physical properties.

Disclosed herein too is a method of manufacturing a guidewire comprising extruding onto a core a jacket, wherein the jacket comprises a segmented layer that is radially disposed upon the core and wherein the segmented layer comprises at least two segments having different compositions and/or different physical properties.

Disclosed herein too is a method of treating a blood vessel or body lumen comprising inserting into the blood vessel or body lumen a guidewire comprising a core; and a jacket disposed upon the core; wherein the jacket comprises a segmented layer that is radially disposed upon the core and wherein the segmented layer comprises at least two segments having different compositions and/or different physical properties; and manipulating the guidewire.

### DETAILED DESCRIPTION OF FIGURES

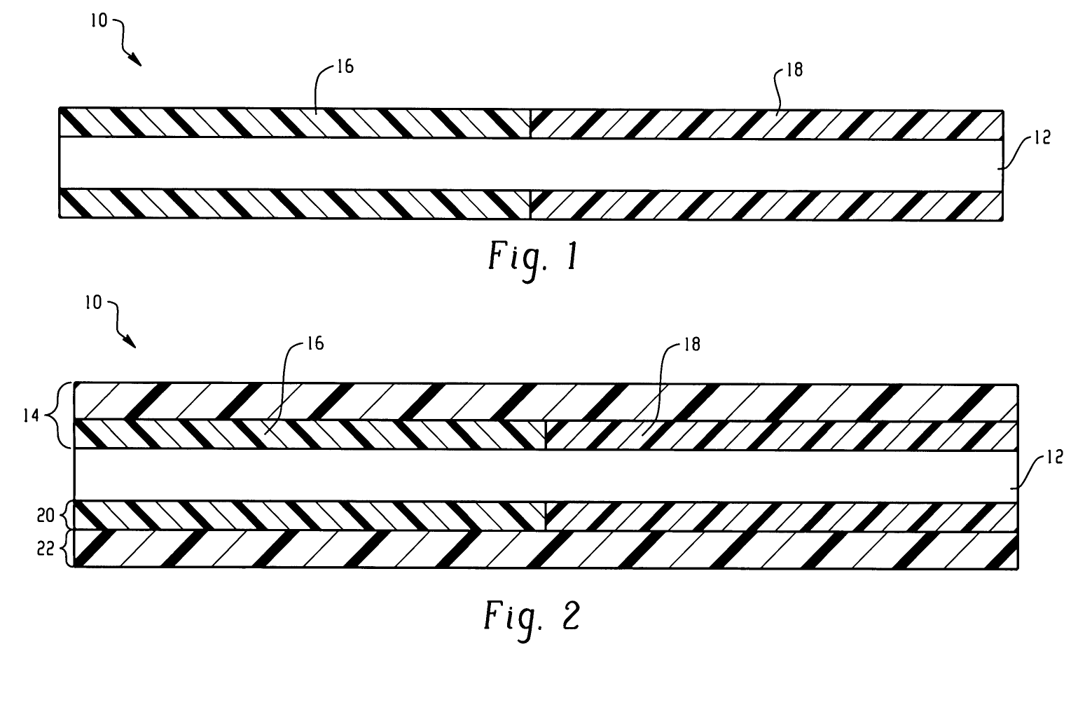

FIG. 1 depicts one embodiment of a guidewire 10 comprising a core 12 upon which is disposed a jacket 14; the jacket comprises a single segmented layer;

FIG. 2 depicts one embodiment of a guidewire 10 comprising a jacket 14 that comprises multiple concentric layers 20 and 22 disposed upon the core 12, wherein one of the layers is a segmented layer having at least two segments;

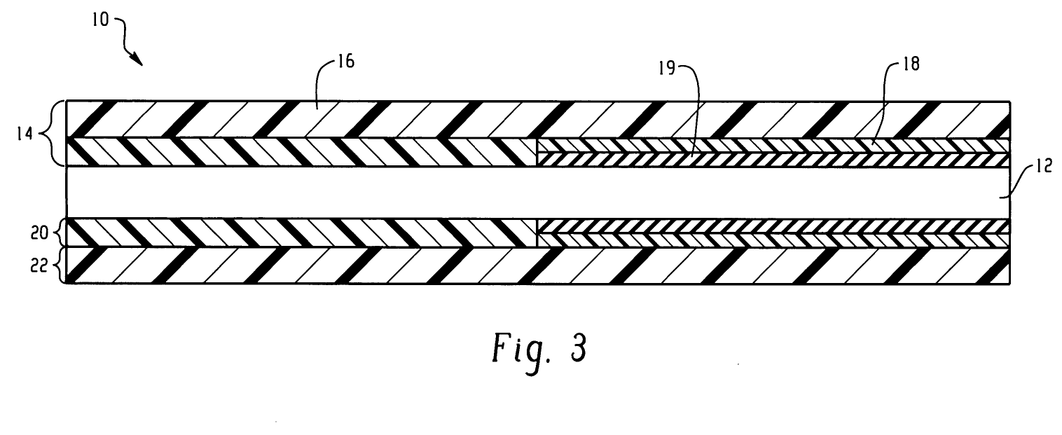

FIG. 3 depicts one embodiment of a guide wire 10 wherein a continuous layer of the jacket 14 is made electrically, thermally or magnetically responsive by the addition of electrically, thermally or magnetically responsive filler so that it can be advantageously used to transmit or react with an electrical current, a magnetic field or thermal source to a segment 16 that is electrically conductive or thermally or magnetically responsive;

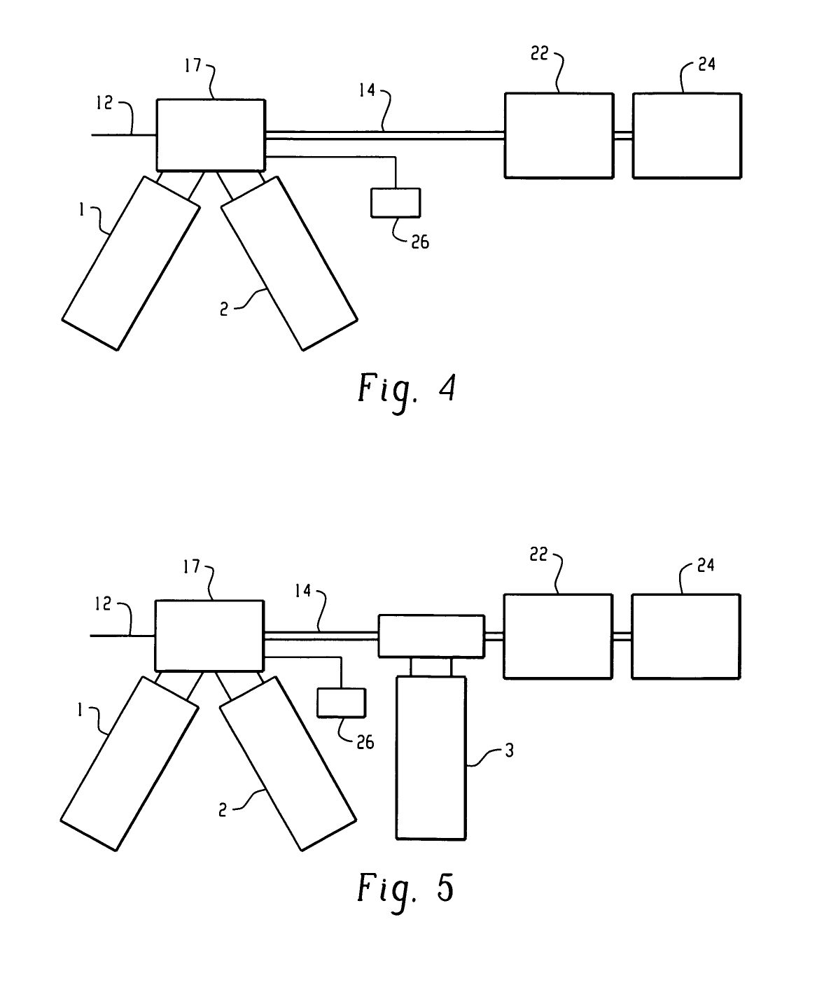

FIG. 4 depicts one exemplary extrusion set-up for disposing a jacket 14 comprising multiple segments onto a core 12; and

FIG. 5 depicts an exemplary extrusion set-up for disposing a multilayered jacket 14 comprising multiple segments onto a core 12.

### DETAILED DESCRIPTION

With reference to the FIG. 1, a guidewire 10 comprises a core 12 upon which is disposed a jacket 14. The jacket 14 comprises a segmented layer. The segmented layer generally comprises polymeric segments that are disposed upon the core 12. In one embodiment, the jacket 14 advantageously comprises a single layer having two segments, a first segment 16 and a second segment 18 manufactured from different polymeric compositions. The different compositions can have different properties if desired. In another embodiment disclosed in the FIG. 2, the jacket 14 advantageously comprises multiple concentric layers 20 and 22 disposed upon the core 12, wherein one of the layers is a segmented layer having at least two segments. In the FIG. 2, the segmented layer comprises a first segment 16 and a second segment 18 manufactured from different polymeric compositions.

The core 12 can be manufactured from a variety and/or combination of different materials. As noted above, the core can be manufactured from metals or plastics. The core 12 generally extends from the proximal end of the guidewire to the distal end. The core 12 can have a cross-section that is tubular, solid, or alternates between tubular and solid. Suitable examples of metals are alloys such stainless steels, shape memory alloys, shape memory alloys that display pseudoelasticity or superelasticity, radio-opaque shape memory alloys, or the like, or a combination comprising at least one of the foregoing metals. Examples of the foregoing metal alloys are 300 and 400 series stainless steel alloys, cobalt-chromium alloys, MP35N or L605 nickel titanium alloys, nickel titanium alloys having radio-opaque ternary elements, nickel free shape memory alloys, beta titanium alloys, or the like, or a combination comprising at least one of the foregoing metal alloys.

Suitable examples of nickel titanium alloys are nickel-titanium-niobium, nickel-titanium-copper, nickel-titanium-iron, nickel-titanium-hafnium, nickel-titanium-palladium, nickel-titanium-gold, nickel-titanium-platinum alloys and the like, and combinations comprising at least one of the foregoing nickel titanium alloys. Preferred alloys are nickel-titanium alloys and titanium-nickel-niobium alloys.

Nickel-titanium alloys that may be used in as the core generally comprise nickel in an amount of about 54.5 wt% to about 57.0 wt% based on the total weight of the alloy. Nickel-titanium-niobium (NiTiNb) alloys that may be used in the medical devices generally comprise nickel in an amount of about 30 wt% to about 56 wt% and niobium in an amount of about 4 wt% to about 43 wt%, with the remainder being titanium. An exemplary composition of a titanium-nickel-niobium alloy is one having about 48 wt% nickel and about 14 wt% niobium, based on the total weight of the alloy.

As noted above, β-titanium alloys can also be used as the core 12. Titanium alloys having a high enough concentration of β stabilizers, generally are sufficiently stable to have a meta-stable β phase structure at room temperature. The alloys showing such a property are called β-titanium alloys. An exemplary β-titanium alloy is one that

comprises an amount of about 8 to about 12 wt% of molybdenum, about 2.8 to about 6 wt% aluminum, up to about 2 wt% vanadium, up to about 4 wt% niobium, with the balance being titanium. All weight percents are based on the total weight of the alloy.

Both the nickel titanium alloys and the β titanium alloys can be made radio-opaque by alloying them with an element that provides radio-opacity to the core 12. Radio opacity permits imaging of the guide wire during treatment of blood vessels inside the body of a patient. Examples of suitable elements that provide radio-opacity to the core 12 are iridium, platinum, gold, rhenium, tungsten, palladium, rhodium, tantalum, silver, ruthenium or hafnium. The guidewire can also be made radio-opaque by coating it with the aforementioned elements that provide it with radio-opacity.

The core 12 can have a cross-sectional geometry that has any desired shape. Examples of suitable cross-sectional geometries are circular, square, triangular, rectangular, polygonal, or the like, or a combination comprising at least one of the foregoing shapes. An exemplary cross-sectional geometry is circular. In one embodiment, the distal end of the guide wire may have a smaller cross-sectional area compared with a portion of the guide wire that is removed from the distal end and closer to the proximal end. This reduction in cross-sectional area of the distal end permits the distal end to have greater flexibility than if the cross-sectional area was not reduced.

If the core 12 is circular, it generally has a diameter of about 0.18 mm to about 0.90 mm. An exemplary diameter is about 0.50 mm.

As noted above, the jacket 14 can comprise a single layer that is segmented or can be multilayered. The segments are manufactured from polymeric materials having differing compositions and/or different properties. The differing compositions permit the use of different functions for each segment. Referring again now to the FIG. 1, the first segment 16 and the second segment 18 can be manufactured from different compositions and therefore have differing stiffnesses. For example, the first segment 16 can have a higher stiffness than the second segment 18. The stiffer segment can be located closer to the distal end of the guide wire than to the proximal end of the guide wire 10 if desired. Alternatively, the stiffer segment can be located closer to the proximal end than to the distal end of the guide wire 10. The location of the stiffer segment can be advantageously used to vary the stiffness and flexibility of the guide wire. By locating a stiffer segment closer to the proximal end of the guide wire and a segment have a lower stiffness closer to the distal end, the flexibility of the distal end of the guide wire can be maintained or even enhanced, when compared with the flexibility of the remainder of the wire.

It is to be noted that the number of segments can vary from 2 to greater than 100 if desired. In one embodiment, at least three segments can be disposed upon the core. In another embodiment, the number of segments disposed upon the core can be greater than or equal to about 5. In another embodiment, the number of segments disposed upon the core can be greater than or equal to about 10.

The length of the segments can be varied. For example, when only two segments are used, the first segment can extend for a length of greater than or equal to about 2% of the total length of the guide wire. In one embodiment, the first segment can extend for a length of greater than or equal to about 10% of the total length of the guide wire. In another embodiment, the first segment can extend for a length of greater than or equal to about 50% of the total length of the guide wire. In yet another embodiment, the first segment can extend for a length of greater than or equal to about 70% of the total length of the guide wire. In yet another embodiment, the first segment can extend for a length equaling about 95% of the total length of the guide wire.

Similarly, when only two segments are used, the second segment can extend for a length of greater than or equal to about 5% of the total length of the guide wire. In one embodiment, the second segment can extend for a length of greater than or equal to about 10% of the total length of the guide wire. In another embodiment, the second segment can extend for a length of greater than or equal to about 50% of the total length of the guide wire. In yet another embodiment, the second segment can extend for a length of greater than or equal to about 70% of the total length of the guide wire. In yet another embodiment, the second segment can extend for a length equaling about 98% of the total length of the guide wire. When more than two segments are used, the length of the respective segments can be varied depending upon the flexibility, torque or force transmittal characteristics desired in the guide wire. A segment can cover the entire surface of the core at a given location if desired. Alternatively a segment can partially cover the surface of the core at a given location.

As noted above, the jacket 14 can be multilayered. In one embodiment, a multilayered jacket 14 comprises a layer that is segmented. In another embodiment, a multilayered jacket 14 comprises a layer that is continuous.

When the guidewire has a multilayered jacket, the respective layers are radially disposed concentrically upon one another such that the outer surface of the first layer is in intimate contact with the inner surface of the second layer. When the jacket is multilayered, at least one of the layers of the jacket is manufactured from an organic polymer or an organic polymeric alloy. A multilayered jacket 14 can have any desirable number of layers. A multilayered jacket can have, for example, two, three, four, five or more layers if desired. As noted above, at least one of the layers comprises at least two segments.

When a multilayered jacket is used, at least one of the layers is segmented. In one embodiment, the segmented layer may be radially disposed directly upon the core, while a continuous layer is radially disposed upon the segmented layer. The term disposed upon indicates that the outer surface of one layer is in intimate contact with the inner surface of another layer. In another embodiment, a continuous layer may be disposed directly upon the core, while a segmented layer is disposed upon and in intimate contact with the continuous layer.

As noted above, at least one layer of the jacket 14 is manufactured from an organic polymer. The organic polymer can be a thermoplastic, a thermoset, or a combination of a thermoplastic with a thermoset. Examples of suitable organic polymers are oligomers, homopolymers, copolymers, block copolymers, graft copolymers, alternating copolymers, star block copolymers, alternating block copolymers, dendrimers, ionic polymers, or the like, or a combination comprising at least one of the foregoing polymers. Examples of suitable polymers that can be used in the jacket 14 are polyarylene sulfides, polyalkyds, polystyrenes, polyesters, polyamides, polyaramides, polyamideimides, polyarylates, polyarylsulfones, polyethersulfones, polyphenylene sulfides, polysulfones, polyimides, polyetherimides, polytetrafluoroethylenes, polyetherketones, polyether etherketones, polyether ketone ketones, polybenzoxazoles, polyoxadiazoles, polybutadienes, polyisoprenes, polybenzothiazinophenothiazines, polybenzothiazoles, polypyrazinoquinoxalines, polypyromellitimides, polyquinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, polyolefins, fluoropolymers, or the like, or a combination comprising at least one of the foregoing organic polymers.

The organic polymers used in the jacket can be modified with fillers and additives to impart properties such as, for example, electrical conductivity, stiffness, frictional properties, adhesive properties, biodegradability, release properties, or the like.

Electrically conductive fillers can be incorporated into the continuous or segmented layers of the jacket 14 in order to heat a section of the core in order to change its stiffness or its shape if desired. Electrically conductive fillers that can be added to the composition are carbon nanotubes, carbon fibers, carbon black, metallic fillers, non-conductive fillers coated with metallic coatings, non-metallic fillers, or the like, or a combination comprising at least one of the foregoing electrically conductive fillers. The electrically conductive fillers can be used in amounts of about 0.01 to about 50 wt%, based on the weight of the layer or segment in which they are utilized. In one embodiment, the electrically conductive fillers are generally used in amounts of about 0.25 wt% to about 30 wt%, based on the total weight of the segment or layer in which they are used. In another embodiment, the electrically conductive fillers are generally used in amounts of about 0.5 wt% to about 10 wt%, based on the total weight of the segment or layer in which they are used. In yet another embodiment, the electrically conductive fillers are generally used in amounts of about 1 wt% to about 5 wt%, based on the total weight of the segment or layer in which they are used.

In one embodiment carbon fibers, VGCF, carbon nanotubes, carbon black, conductive metal fillers, conductive non-metal fillers, metal coated fillers as detailed above, or any combination of the foregoing may be used in the segment or layer to render it electrically conductive. Exemplary electrically conductive fillers are carbon nanotubes. It is generally desirable to use the conductive fillers in amounts effective to produce surface resistivity less than or equal to about 10⁹ ohm/square as measured as per ASTM D 257. In another embodiment, it is desirable of have the surface resistivity of the thermoplastic composition be less than or equal to about 10⁷ ohm/square. In yet another embodiment, it is desirable of have the surface resistivity of the thermoplastic composition be less than or equal to about 10⁵ ohm/square. It is also desirable to have the volume resistivity less than or equal to about 10¹² ohm-centimeter. In one embodiment, it is desirable to have the volume resistivity less than or equal to about 10⁶ ohm-centimeter. In another embodiment, it is desirable to have the volume resistivity less than or equal to about 10³ ohm-centimeter. In yet another embodiment, it is desirable to have the volume resistivity less than or equal to about 100 ohm-centimeter.

In one embodiment, related to the addition of conductive fillers to a segment or a continuous layer, an electrical current can be supplied to the jacket so as to resistively promote heating in the jacket. The heat can be used to change the temperature of the jacket and/or the core in order to promote a change in stiffness of the core. FIG. 3 depicts an embodiment of a guide wire 10 wherein a continuous layer of the jacket 14 is made electrically conductive by the addition of an electrically conductive filler so that it can be advantageously used to transmit an electrical current to a segment 16. The segment 16 is also heated resistively, and this heating can be used to facilitate a change in the shape or stiffness of the core 14. In this application, an insulating layer or segment 19 is disposed between the segment 16 and the core 14. An optional insulating layer can also disposed on the outer surface of the continuous layer if desired.

In another embodiment, a magnetizable filler may be added to a segment or a continuous layer, so that it can respond to an applied magnetic field or to an electrical field. Examples of specific magnetizable particles are particles comprised of materials such as iron, iron oxide, iron nitride, iron carbide, silicon steel, nickel, cobalt, low carbon steel, carbonyl iron, chromium dioxide, or the like, or a combination comprising at least one of the foregoing. When a magnetizable filler is added to a segment, the stiffness of the segment can be changed by applying a magnetic field to the segment. The applied magnetic field can promote a change in orientation of the fillers in the segment. The stiffness of the segment may be changed by applying an electrical field to the segment. The electric current may be transmitted by the continuous layer.

In yet another embodiment, fillers that have a high thermal conductivity may be added to a segment or a continuous layer. The addition of such fillers in conjunction with electrically conductive fillers will promote heating of a segment and facilitate a change in the stiffness of the segment. Fillers that can respond to an ultrasonic stimulus can also be added to the segment or to the continuous layer.

The stiffness of the segments or the continuous layers used in the jacket 14 can be varied by using the aforementioned fillers. As noted above, it is desirable to have certain segments having a lesser stiffness than certain other segments. The stiffness of the segments can be increased by the addition of fillers. There is no particular limitation to the shape of the particles, which may be for example, spherical, irregular, plate-like or whisker like. The fillers can be nanometer sized particles or micrometer sized particles. The nanometer sized particles may generally have average largest dimensions of less than or equal to about 200 nanometers (nm). In one embodiment, the particles may have average largest dimensions of less than or equal to about 150 nm. In another embodiment, the particles may have average largest dimensions of less than or equal to about 100 nm. In yet another embodiment, the particles may have average largest dimensions of less than or equal to about 75 nm. In yet another embodiment, the particles may have average largest dimensions of less than or equal to about 50 nm. As stated above, the nanosized particles may generally have average largest dimensions of less than or equal to about 200 nm. In one embodiment, more than 90% of the particles have average largest dimensions less than or equal to about 200 nm. In another embodiment, more than 95% of the particles have average largest dimensions less than or equal to about 200 nm. In yet another embodiment, more than 99% of the particles have average largest dimensions less than or equal to about 200 nm. Bimodal or higher particle size distributions may be used. The micrometer sized particles are generally those having average largest dimensions of greater than or equal to about 200 nanometers.

In another embodiment, the jacket 14 can comprise a segment that is in the form of a foam and contains a biologically active agent in the pores of the foam. The biologically active agent can be released from the pores to treat the blood vessel. In one embodiment, the biologically active agent can be released upon the application of heat to the foam. In another embodiment, pressure can be used to release the biologically active agent into the blood vessel.

In another embodiment, at least one of the segments is manufactured from a material that is reversibly expandable. Thus upon applying an electrical stimulus or a thermal stimulus, the segment can undergo expansion. In one embodiment, upon removing the electrical or thermal stimulus, the segment can begin to contract. In another embodiment, upon continued application of the electrical or thermal stimulus, the segment will begin to contract. Segments that can reversibly expand can be used to affix the position of the guidewire during treatment of the blood vessel. By expanding the segment during surgery, the guidewire can be affixed while the treatment of the blood vessel is being accomplished. After the treatment is completed, the segment is allowed to contract, thereby facilitating the removal of the guidewire from the blood vessel.

In another embodiment, a segment can be manufactured from a shape memory polymer. The segment can then return to its predetermined shape upon the application of a thermal stimulus. A segment manufactured from a shape memory polymer can also be used to affix the guidewire 10 to a blood vessel during surgery. In one embodiment, a segment can communicate with another segment. Thus the flexibility of one segment can be controlled by another segment. The communication between segments can be mechanical, electrical, magnetic, or electro-mechanical.

In yet another embodiment, a layer of hot melt adhesive can be applied between certain segments of the jacket 14 and the core 12. The layer of hot melt adhesive can be applied to the core 12 prior to the extrusion of the segment onto core 12. The hot melt adhesive layer will prevent any motion between the core 12 and the segment in those regions where the adhesive is applied. In other regions, the lack of adhesion between the segment and the core will permit motion between the core and the jacket. In one embodiment, the outer surface of the core 12 can be textured to provide mechanical adhesion between the jacket 14 and the core 12.

In one embodiment, a segment that is disposed upon the core and located closer to the proximal end is provided with an adhesive layer that prevents relative motion between the segment and the core 12. An adjacent segment disposed upon the core and located closer to the proximal end is not provided with such an adhesive layer and permits relative motion between the segment and the core. Thus the segment with the applied adhesive can be used to permit sliding and/or rotary motion between the segment without the applied adhesive and the core.

As noted above, when the jacket 14 is multilayered, the segmented layer can be disposed upon the core 12, while the continuous layer is radially disposed upon the segmented layer. In one embodiment, the continuous layer can be disposed upon the core 12, while the segmented layer is radially disposed upon the continuous layer.

The segmented layer of the jacket 14 can be disposed upon the core 12 in an extrusion process by using an extrusion apparatus depicted in FIG. 4. This process is disclosed in U.S. Pat. No. 4,888,146 to Dandeneau, the entire contents of which are hereby incorporated by reference. In the FIG. 4, the core 12 traverses an extrusion head 17, wherein the extrusion head 17 is regulated by a control device 26 such that a first organic polymer and a second organic polymer that are respectively extruded from extruders 1 and 2 is progressively fed through an extrusion head 17 onto the core 12. The core 12 along with the segmented layers disposed upon the core 14 is directed into a cooling trough or station 22 and then to a wind-up reel or station 24.

When the guidewire 10 comprises a multilayered jacket, the core 12 can be subjected to a second extrusion process so as to dispose multiple layers on the core 12. FIG. 5 depicts one exemplary embodiment by which a multilayered jacket can be disposed upon a core 12. In the FIG. 5, the core 12 is first fed through the extrusion head 17 depicted in FIG. 4, whereupon a segmented layer is disposed upon the core 12. Following the deposition of the segmented layer, the guidewire 10 is subjected to a second extrusion step in an extruder 3 to dispose a second layer, third layer or a fourth layer on the guidewire 10. The deposition of multiple layers can be accomplished in a single continuous process or in a batch process. An exemplary extrusion processes in which a multilayered jacket can be disposed upon the core 12 is by using crosshead extrusion as disclosed in U.S. Pat. No. 6,447,835, the entire contents of which are hereby incorporated by reference. For example, after the first segmented layer is disposed upon the core 12, the guidewire can be directly subjected to a second extrusion process wherein a continuous layer is disposed upon the segmented layer to form the jacket 14.

Alternatively, a continuous layer may first be disposed upon the core 12 in a crosshead extrusion process, followed by which a segmented layer can be disposed upon the continuous layer by utilizing the process described in U.S. Pat. No. 4,888,146 and depicted in the FIG. 4.

The disclosed guidewire 10 can be employed during surgery in a variety of methods. In one embodiment, when the guidewire 10 has disposed upon it electrically conductive segments, an electrical current can be used to resistively heat a portion of the guidewire 10 to change the stiffness or shape of the core.

In another embodiment, when the jacket 14 comprises a foamed layer or a foamed segment that is porous, a biologically active agent can be transmitted to a desired site in a blood vessel, in order to facilitate treatment of the blood vessel.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

What is claimed is:

1. A guidewire comprising:
a core; and
a jacket disposed upon the core; wherein the jacket comprises a segmented layer that is radially disposed upon the core and wherein the segmented layer comprises at least two segments having different compositions and/or different physical properties.

2. The guidewire of claim 1, wherein the jacket surrounds the core and is in intimate contact with the core.

3. The guidewire of claim 1, wherein the core comprises a metal or an organic polymer.

4. The guidewire of claim 3, wherein the metal is a metal alloy and wherein the alloy is a stainless steel alloy, a cobalt-chromium alloy, a shape memory alloy, a shape memory alloy that display pseudoelasticity or superelasticity, a radio-opaque shape memory alloy, or a combination comprising at least one of the foregoing metals.

5. The guidewire of claim 3, wherein the metal alloys are 300 or 400 series stainless steel alloys, MP35N or L605 cobalt-chromium alloys, nickel titanium alloys, nickel titanium alloys having radio-opaque ternary elements, nickel free shape memory alloys, beta titanium alloys, or a combination comprising at least one of the foregoing metal alloys.

6. The guidewire of claim 1, wherein the jacket comprises an organic polymer.

7. The guidewire of claim 1, wherein the jacket is multilayered.

8. The guidewire of claim 1, wherein the jacket further comprises a continuous layer that is in physical contact with a segment of the segmented layer.

9. The guidewire of claim 8, wherein the continuous layer and the segment of the segmented layer is reversibly expandable, electrically conducting, magnetically susceptible, thermally conductive and/or porous.

10. The guidewire of claim 6, wherein the organic polymer is a thermoplastic, a thermoset or a combination of a thermoplastic with a thermoset.

11. The guidewire of claim 6, wherein the organic polymer is an oligomer, a homopolymer, a copolymer, a block copolymer, a graft copolymer, an alternating copolymer, a star block copolymer, an alternating block copolymer, a dendrimer, an ionic polymer, or a combination comprising at least one of the foregoing polymers.

12. The guidewire of claim 6, wherein the organic polymer is a polyarylene sulfide, a polyalkyd, a polystyrene, a polyester, a polyamide, polyaramides, a polyamideimide, a polyarylate, a polyarylsulfone, a polyethersulfone, a polyimide, a polyetherimide, a polytetrafluoroethylene, a polyetherketone, a polyether etherketone, a polyether ketone ketone, a polybenzoxazole, a polyoxadiazole, a polybenzothiazinophenothiazine, a polybenzothiazole, a polypyrazinoquinoxaline, a polypyromellitimide, a polyquinoxaline, a polybenzimidazole, a polyoxindole, a polyoxoisoindoline, a polydioxoisoindoline, a polytriazine, a polypyridazine, a polypiperazine, a polypyridine, a polypiperidine, a polytriazole, a polypyrazole, a polycarborane, a polyoxabicyclononane, a polydibenzofuran, a polyphthalide, a polyacetal, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polysulfide, a polysulfonamide, a polyurea, a polyphosphazene, a polysilazane, a polyolefin, a polysiloxane, or a combination comprising at least one of the foregoing thermoplastic polymers.

13. The guidewire of claim 1, wherein the segmented layer comprises a first segment having a first stiffness and a second segment having a second stiffness.

14. The guidewire of claim 13, wherein the first segment is stiffer than the second segment, and wherein the first segment is located closer to the proximal end of the guide wire than the distal end.

15. The guidewire of claim 13, wherein the first segment is stiffer than the second segment, and wherein the first segment is located closer to the distal end of the guide wire than the proximal end.

16. The guidewire of claim 1, wherein one of the segments is electrically conducting, porous and/or reversibly expanding.

17. The guidewire of claim 1, wherein the first segment is in mechanical, thermal or electrical communication with the second segment.

18. A method of manufacturing a guidewire comprising:
extruding onto a core a jacket, wherein the jacket comprises a segmented layer that is radially disposed upon the core and wherein the segmented layer comprises at least two segments having different compositions and/or different physical properties.

19. The method of claim 18, wherein the core is a tube, a wire, or a cylinder or a combination thereof.

20. The method of claim 18, further extruding onto the core a continuous layer to form a multilayered jacket, wherein the continuous layer is radially disposed upon the segmented layer.

21. The method of claim 20, wherein the extrusion of the continuous layer is accomplished by crosshead extrusion.

22. A method of treating a blood vessel comprising:
inserting into the blood vessel a guidewire comprising:
a core; and a jacket disposed upon the core; wherein the jacket comprises a segmented layer that is radially disposed upon the core and wherein the segmented layer comprises at least two segments having different compositions and/or different physical properties; and
manipulating the guidewire.

23. The method of claim 22, wherein treating the blood vessel comprises changing a property of one segment using an electrical stimulus or a thermal stimulus.

24. The method of claim 22, wherein treating the blood vessel comprises releasing a biological agent from one segment.

25. The method of claim 22, wherein one segment is immovable relative to the core while another segment can move relative to the core.
